(19) United States
(12) Patent Application Publication
(10) Pub. No.: US 2026/0021040 A1
(43) Pub. Date: Jan. 22, 2026

(54) CHEMICAL COMPOSITION FOR ORAL APPLICATIONS

(71) Applicant: NovoMedTek LLC, Jacksonville, FL (US)

(72) Inventor: Ethan Glenn Sherman, Jacksonville, FL (US)

(21) Appl. No.: 19/275,496

(22) Filed: Jul. 21, 2025

Related U.S. Application Data
(60) Provisional application No. 63/673,961, filed on Jul. 22, 2024.

(51) Int. Cl. A61K 9/00; A61J 7/00; A61K 9/14; A61K 31/4425; A61K 47/36; A61K 47/42

(52) U.S. Cl. CPC A61K 9/006; A61J 7/0053; A61K 9/14; A61K 31/4425; A61K 47/36; A61K 47/42

(57) ABSTRACT

A powdered composition for oral application of therapeutic agents includes a base material, at least one therapeutic agent, and a crosslinker. The composition is formed as a homogenous powder. An application device is also disclosed.

### CROSS-REFERENCE TO RELATED APPLICATION DATA

This application claims the benefit of and priority to Provisional U.S. Patent Application Ser. No. 63/673,961, filed, Jul. 22, 2024, titled CHEMICAL COMPOSITION FOR ORAL APPLICATIONS, the disclosure of which is incorporated herein in its entirety.

### BACKGROUND

The present disclosure relates to chemical compositions, and more particularly, to a powdered chemical composition for the retention of oral medicinal compounds in applications, such as oral applications.

Treatment of periodontal conditions such as periodontitis and peri-implantitis traditionally requires application of a liquid, gel, film, or drug encapsulated microsphere to the periodontal pocket in order to help reduce bioburden, pain, sensitivity, and to aid in wound healing. Custom gels or solutions can be compounded to treat a variety of periodontal conditions, such as periodontitis, peri-implantitis, inflammation, infection, and/or for pain management. However, these liquids, gels, films, and microspheres have limited adhesion and short retention time in the oral cavity due to poor adhesion to mucosal surfaces.

Accordingly, there is a need for a chemical composition for oral application that can increase mucosal retention of periodontal treatments.

### SUMMARY

In one aspect, a powdered composition for oral application of therapeutic agents includes a base material, at least one therapeutic agent, and a crosslinker. The composition is formed as a homogenous powder. In embodiments, the base material is one or a combination of a polysaccharide, collagen, chitosan, agar, a thermopolymer, poloxamer, xanthium gum, alginic acid, cellulose, and sodium alginate. The material can be, for example, a polymer, such as a polysaccharide, collagen, alginate, chitosan, and combinations thereof. In embodiments, the base material is present in a concentration of about 75 percent to about 99 percent by weight of the composition, and in a concentration of about 90 percent to about 95 percent by weight of the composition.

The therapeutic agent is one or a combination of antimicrobial surfactants, hemostats, antibiotics, and anti-inflammatory agents. In embodiments, the antimicrobial surfactant can be, for example cetylpyridinium chloride. The cetylpyridinium chloride can be present, for example, in a concentration of about 0.010 percent to about 0.10 percent by weight of the composition.

In embodiments, the crosslinker is a homo-bifunctional, a hetero-bifunctional, photoreactive crosslinking agent or a combination thereof. For example, a homo-bifunctional crosslinker can be a divalent metal ion crosslinker, such as one or a combination of calcium sulfate and calcium chloride. In embodiments, the calcium crosslinker is present in a concentration about 1.0 percent to about 10.0 percent by weight of the composition. In embodiments, the calcium crosslinker is present in a concentration of about 5.21 percent by weight of the composition.

In an aspect, the composition includes a polymer base material present in concentration of about 90 percent to about 95 percent by weight of the composition, a crosslinker present in a concentration of about 5 percent to about 10 percent by weight of the composition, and an antimicrobial therapeutic agent present in a concentration of about 0.01 percent to about 0.10 percent by weight of the composition.

In another aspect, a powdered composition for oral application includes a base material and a crosslinker. The composition is formed as a homogenous powder. The composition, when subject to a moist anatomical location, hydrates into a mucoadhesive or bioadhesive hydrogel. The mucoadhesive or bioadhesive gel can crosslink in-situ into a solid hydrogel. The solid hydrogel can form a barrier to microbial contamination.

In another aspect, a delivery device for a powdered composition for oral application of a therapeutic agent includes a syringe including a body, a syringe plunger rod, a dispenser hub, a dispenser tip, a cap, a composition plunger rod and syringe plunger tip. The powdered composition can be stored in the dispenser tip, and depressing the syringe plunger rod dispenses the powdered composition from the delivery device. In embodiments, the delivery device is disposable in its entirety.

In embodiments, the dispenser hub is a Luer dispenser hub, the dispenser tip is a Luer dispenser tip and fastens to the Luer dispenser hub.

The foregoing general description and the following detailed description are examples only and are not restrictive of the present disclosure.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 is a flow diagram of a process for manufacture and delivery of a chemical composition for oral applications, according to an embodiment of the present disclosure;

FIG. 2 is a graph of the mass in grams (g) of swelled composition vs. time (in hours) of the composition submerged;

FIG. 3 is a graph of the dissolution rate of the composition at high, medium, and low cross-linker concentrations, vs. time (in days);

FIG. 4 is a schematic diagram of a test device used to test the adhesion (measuring shear stress) of a sample treated with the composition;

FIG. 5 is a chart of the adhesion of a sample treated with the composition and samples treated with other periodontal treatment compositions;

FIG. 6 is another chart of the adhesion of a sample treated with the composition and samples treated with other periodontal treatment compositions;

FIGS. 7A-7D are photographs of samples prepared for (FIG. 7A) and following testing (FIGS. 7B-7D) for bacterial growth following application of the present composition and samples treated with other periodontal treatment compositions;

FIG. 8 is a chart illustrating the results of the bacterial growth testing as measured by the size of the zone of inhibition (ZOI) exhibited;

FIG. 9 is another chart illustrating the results of the bacterial growth testing for submerged specimens as measured by the size of the zone of inhibition (ZOI) exhibited vs. time (in days submerged);

FIG. 10 is a still another chart illustrating the results of the bacterial growth testing as measured by the size of the zone of inhibition (ZOI) exhibited vs. time (in days) and with hydrated samples; and

FIGS. 11A-11D are various views of an embodiment of an oral powder delivery device.

### DETAILED DESCRIPTION

The following detailed description is of the best currently contemplated modes of carrying out exemplary embodiments of the present disclosure. The description is not to be taken in a limiting sense but is made merely for the purpose of illustrating the general principles of the present disclosure, since the scope of the present disclosure is best defined by the appended claims.

Existing periodontal treatments to reduce bioburden, pain, and sensitivity have limited retention in the oral cavity due to poor adhesion and retention to mucosal surfaces. Broadly, an embodiment of the present disclosure provides a chemical composition of a powdered polymer that crosslinks in-situ into a hydrogel increasing adhesion and prolonging residence on mucosal surfaces providing prolonged therapeutic efficacy. Specifically, the present disclosure improves adhesion of oral wound dressings, and when hydrated, crosslinks into a hydrogel, further increasing therapeutic contact and retention time. The composition of the present subject matter may also be used as a stent to prevent tissue from adhering, as a hemostatic wound dressing when combined with hemostatic agents, and as a wound dressing for topical wounds.

It will be appreciated that the therapeutic efficacy of such a composition and its application resides in three basic characteristics, namely, contact, coverage, and concentration. That is, the composition should fully or nearly fully contact the area or areas in need, the coverage of the area should be as complete as possible, and the concentration of the composition should be as high or as high as possible within appropriate limits. This is referred to as “the three Cs”.

The present disclosure includes at least three components including a polymer base, at least one therapeutic agent, and a crosslinker. The concentration of each component may be optimized to enhance the adhesion, therapeutic delivery, or crosslinking rate.

Advantageously, the polymer can be adapted to rapidly hydrate with polar liquids, such as water, into a hydrogel that adheres to a mucosal surface. In embodiments, the polymer base can be a polysaccharide such as collagen, chitosan, agar, a thermopolymer (such as poloxamer), xanthium gum, alginic acid, and/or cellulose. In some embodiments, multiple polymers may be used to provide additional performance characteristics, such as scaffolding. For example, sodium alginate (CAS #9005-38-3) may be used. The polymer base may be provided in any suitable amount, for example greater than 50 weight %, in a range of 75-99, preferably about 90 to 95 weight %, e.g., about 94.78 weight %.

In embodiments, adapting the polymer chosen for rapid hydration can be achieved by modifying the particle size of the polymer. In embodiments, size modification can be achieved by grinding, sifting, or spray drying the polymer to reduce the particle size. Preferably, the powder composition is homogeneous with a uniform particle size. In embodiments, the particle diameter can be between 1-100 μm, depending on the polymer chosen as the base. In embodiments, the polymer base remains as an inactive dry powder prior to hydration. Advantageously, the polymer base can achieve improved wet tissue adhesion by rapid absorption of interfacial water due to the reduced surface area of the particles and the use of the polymer base with high solubility in polar liquids.

In embodiments, self-aggregation of the polymer chains forms a colloidal network which displaces the interfacial water on the tissue surface increasing adhesion. Advantageously, adhesion is further enhanced by hydrogen bonding between a moist surface, such as mucosal surfaces, and the powder promoting tissue adhesion.

In embodiments, any number of therapeutic agents may be incorporated into the polymer base to provide at least one therapeutic effect. In preferred embodiments, at least one therapeutic agent is readily solubilized in polar solutions and biocompatible. Advantageously, at least one therapeutic agent elutes into surrounding tissue and the polymer hydrogel degrades via hydrolysis or by metal ion substitution, and/or bio-fragmentation, promoting wound healing. Metal ion substitution refers to, for example, replacement of calcium with sodium in a cross-linked calcium alginate network, thus causing the gel to dissolve. Bio-fragmentation refers to, for example, the breaking apart of gel into smaller parts vis enzymes excreted by microorganisms that reduce the molecular weight of the material making it more easily absorbed into the body. The degraded subcomponents may be readily absorbed into and/or digested/metabolized by the subject's body.

In embodiments, at least one therapeutic effect can be adapted to provide at least one of: prevention of microbial contamination, reduction of inflammation, medicinal materials such as antibiotics, and/or provision of hemostasis depending on the chosen anatomical application. For example, antimicrobial surfactants, hemostats, or anti-inflammatory agents are common therapeutic agents that can be readily incorporated into hydrogels for delivery to tissue. The therapeutic agents may be provided in any suitable amount. For example, an antimicrobial surfactant such as cetylpyridinium chloride (CPC) may be included in an amount of between about 0.010 to 0.10 weight %.

In some embodiments, the polymer base may be produced without a therapeutic agent. For example, a therapeutic agent may be absent if the hydrogel is applied merely to maintain hydration of tissue or to provide a protective barrier for a wound. Alternatively, the therapeutic agent may be added to the polymer base separately prior to application.

In embodiments, at least one crosslinker can be provided to promote in-vivo residence of hydrogel colloidal networks and prolonged efficacy of therapeutic agents. In embodiments, at least one crosslinker can be homo-bifunctional, hetero-bifunctional, or photoreactive crosslinking reagents. Specifically, homo-bifunctional crosslinkers such as divalent metal ion crosslinkers are capable of rapidly and, in some cases, reversibly crosslinking polymer networks in solution. As an example, in embodiments, the at least one crosslinker can be a calcium crosslinker such as calcium sulfate, CaSO4, or calcium chloride (CaCl, CAS #10043-52-4). The crosslinker may be provided in any suitable amount effective to crosslink the polymer, such as about 1.0 to about 10.0 weight %, for example, 5.21 weight %.

Referring now to the application of at least one crosslinker to the polymer base, at least one crosslinker may be added to the dry powder polymer base. In embodiments, prior to hydration, the at least one crosslinker remains inactive. In some embodiments, the polymer and crosslinker may solubilize before the therapeutic agent, allowing the hydrogel to rapidly form and adhere to the tissue surface. Alternatively, in some embodiments, the polymer and therapeutic agent may solubilize before the crosslinker, allowing the hydrogel to rapidly hydrate and release the therapeutic agent, while allowing the crosslinker to adhere the gel to the tissue surface and increase retention time. The therapeutic agent may elute from the hydrogel in an extended-release manner, for example, if the solubility of the therapeutic agent is lower than the solubility of the polymer and the crosslinker. In embodiments, a ratio of the polymer base to at least one crosslinker promotes rapid tissue adhesion while at least one crosslinker hydrates and strengthens the underlying polymer base, preventing premature removal from the tissue surface.

In addition, the cross-linker can be provided by the surrounding environment, such as liquid present in anatomical locations such as saliva in the oral cavity, mucus in the nasal sinuses, blood in wounds, vaginal secretions, and the like. These anatomical environments provide solutions that contain metal ions, e.g., calcium, magnesium, iron, and the like, that can be used to crosslink the polymer network after application to an anatomic site without direct incorporation of the metal ions during powder manufacture.

Various tests were conducted to determine the efficacy of the present composition. The tests include hydration and swelling testing, hydration/dissolution testing, adhesion testing, microbial barrier testing, and antimicrobial zone of inhibition (ZOI) testing.,

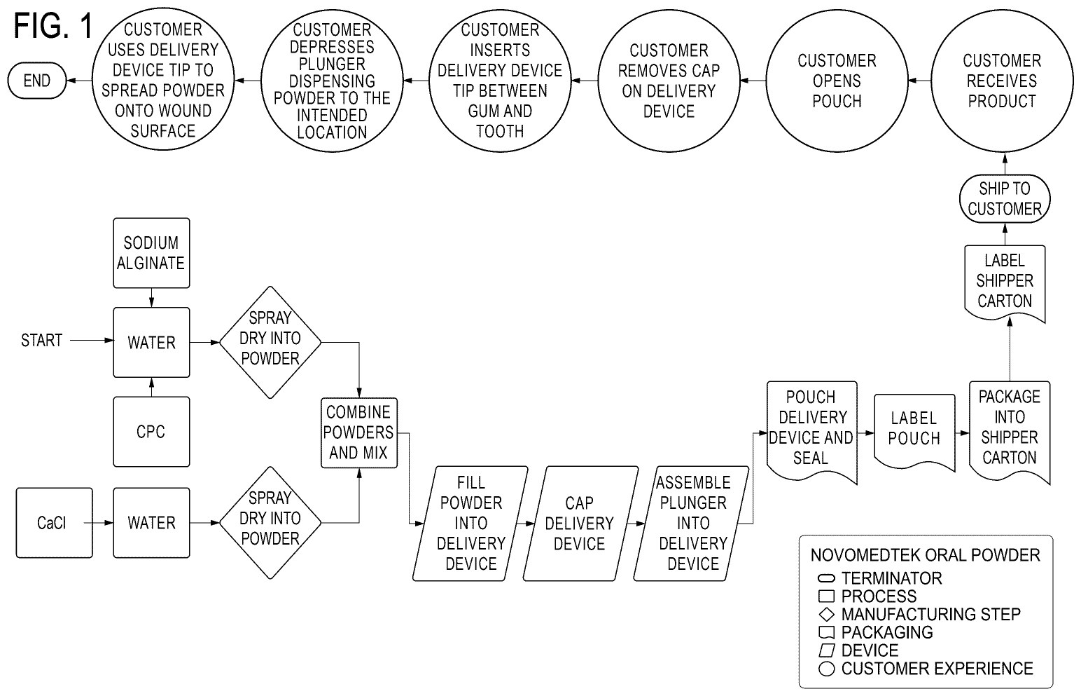

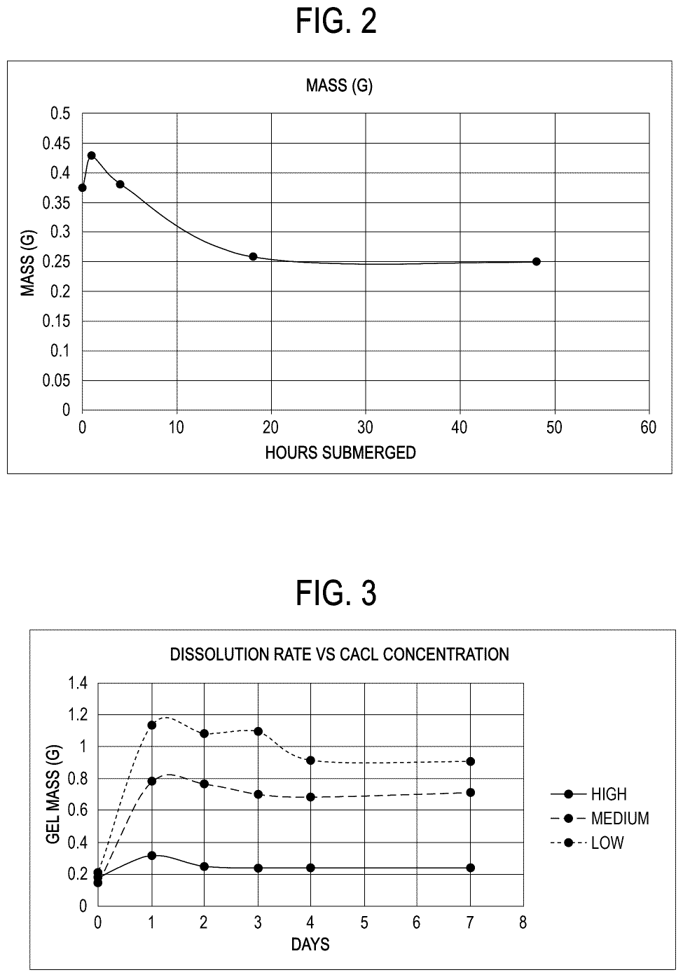

For the hydration and swelling testing, a dry powder of a composition of 90-95% by weight of the polymer base, 5-10% by weight of the crosslinker and 0.01-0.1% of an antimicrobial therapeutic agent was prepared by mixing the polymer base and antimicrobial therapeutic agent in a polar solvent (water) until solubilized and then spray drying the solution into a homogeneous powder. Subsequently, the crosslinker is also hydrated in a polar solvent (water) until solubilized and then spray dried into a homogeneous powder. Both powders were mixed together in the ratios noted above. This process is illustrated in FIG. 1. The dry powder was hydrated with water and allowed to crosslink into a gel. The gel was then measured and submerged into water over time. In this test, the swelling nature of the hydrated powder as it forms a gel over time was measured. The length, width, area, and mass measurements are as measured for the sample tested. The results of these tests are shown in Table 1, below and graphically in FIG. 2.

TABLE 1
Hydration and Swelling of Composition Samples vs. Time

| | Time | Length | Width | Area | Mass |
|---|---|---|---|---|---|
| Test | (hrs) | (in/mm) | (in/mm) | (in²/mm²) | (g) |
| 1 | 0 | 0.39/10 | 0.39/10 | 0.122/78.54 | 0.375 |
| 2 | 1 | 0.55/14 | 0.43/11 | 0.186/120.05 | 0.429 |
| 3 | 4 | 0.47/12 | 0.43/11 | 0.161/103.67 | 0.381 |
| 4 | 18 | 0.43/11 | 0.35/9 | 0.121/77.75 | 0.258 |
| 5 | 48 | .043/11 | 0.35/9 | 0.121/77.75 | 0.25 |

As can be seen from the data of Table 1 and FIG. 2, the gel initially increases in mass as it absorbs water and once crosslinking has occurred, the swelling levels off and remains stable.

Hydration/dissolution testing was conducted to evaluate the impact of crosslinking concentration on the hydration and swelling rate of the gel. The results of this test are shown in Table 2, below and graphically in FIG. 3. From the data (and included graph) it was decided that the “High” concentration was to be used in testing moving forward due to its dissolution properties (ability to dissolve away) over time.

TABLE 2
Hydration/Dissolution Testing of Various
Compound Formulations Over Time

| Test | Configuration | Configuration | % CaCl | Mass |
|---|---|---|---|---|
| Day | FR | Type (CaCl [ ]) | Crosslinker | (g) |
| 0 | FR-24-05-002 | High | 5.2 | 0.183 |
| 0 | FR-24-06-001 | Medium | 2.06 | 0.146 |
| 0 | FR-24-05-004 | Low | 1.09 | 0.212 |
| 1 | FR-24-05-002 | High | 5.2 | 0.313 |
| 1 | FR-24-06-001 | Medium | 2.06 | 0.781 |
| 1 | FR-24-05-004 | Low | 1.09 | 1.135 |
| 2 | FR-24-05-002 | High | 5.2 | 0.248 |
| 2 | FR-24-06-001 | Medium | 2.06 | 0.766 |
| 2 | FR-24-05-004 | Low | 1.09 | 1.082 |
| 3 | FR-24-05-002 | High | 5.2 | 0.239 |
| 3 | FR-24-06-001 | Medium | 2.06 | 0.701 |
| 3 | FR-24-05-004 | Low | 1.09 | 1.095 |
| 4 | FR-24-05-002 | High | 5.2 | 0.238 |
| 4 | FR-24-06-001 | Medium | 2.06 | 0.684 |
| 4 | FR-24-05-004 | Low | 1.09 | 0.913 |
| 7 | FR-24-05-002 | High | 5.2 | 0.239 |
| 7 | FR-24-06-001 | Medium | 2.06 | 0.712 |
| 7 | FR-24-05-004 | Low | 1.09 | 0.906 |

In Table 2, above, different composition formulations (FR) were prepared varying the concentration of a cross-linking agent (CaCl) in a high concentration at 5.2% by weight of the composition, a medium concentration at 2.06% by weight of the compound and a low concentration at 1.09% by weight of the composition, and shows the mass in grams of the composition in days after exposure. This data shows that at high concentrations of cross-linking agent, the mass of the composition decreases (the composition dissolves), which for certain uses can be beneficial once the effectiveness of any medicinal agents or the protective nature of the composition are no longer required.

As noted above, maximum contact and coverage of the composition is desirable for optimum effectiveness of the composition. Adhesion testing was conducted to evaluate the adhesive strength or residence of the powder upon application, that is how well the powder remains in place once applied to the tissue-like substrate after hydration.

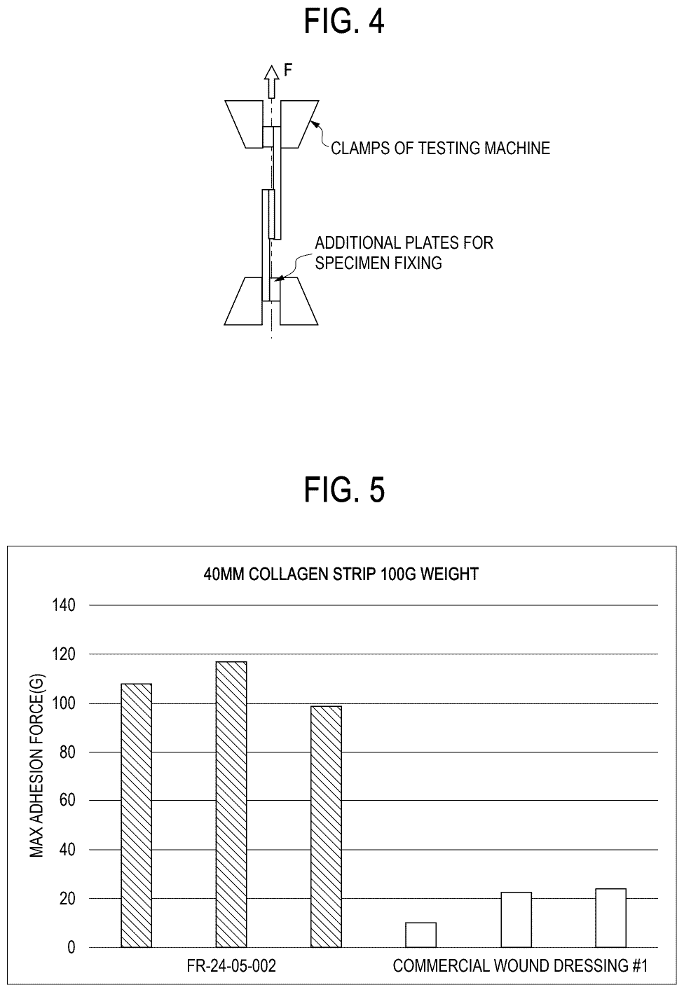

In conducting this test, the powdered composition was applied onto a hydrated collagen sheet (porcine collagen casing) 40 mm wide. The powder was applied to a bottom sheet and was covered with an equivalent 40 mm wide hydrated collagen sheet overlapped in a lap-sheer test fixture, the configuration of which is illustrated in FIG. 4.

The collagen “sandwich” was compressed together using a 100 g weight to press out any air gaps and to produce a uniform gel test specimen. The powder was allowed to hydrate for about 30 minutes using the moisture in the hydrated collagen sheet prior to tensile testing. The powder was allowed to hydrate completely and then placed into the test fixture to measure tensile force. The samples were pulled apart and the maximum tensile force was recorded. The results were compared to a commercially available oral hydrogel wound dressing (commercially available oral hydrogel wound dressing #1). This test was conducted using the FR-24-05-002 indicated above, and the results are shown in Table 3, below and graphically in FIG. 5.

TABLE 3
Adhesion testing of Composition FR-24-05-002 vs.
Commercially Available Oral Wound Dressing Product

| | | Max Adhesion |
|---|---|---|
| Test | Product | Force (g) |
| 1 | FR-24-05-002 | 108 |
| 2 | | 117 |
| 3 | | 99 |
| 1 | Commercially | 10 |
| 2 | available oral | 23 |
| 3 | hydrogel wound | 24 |
| | dressing #1 | |

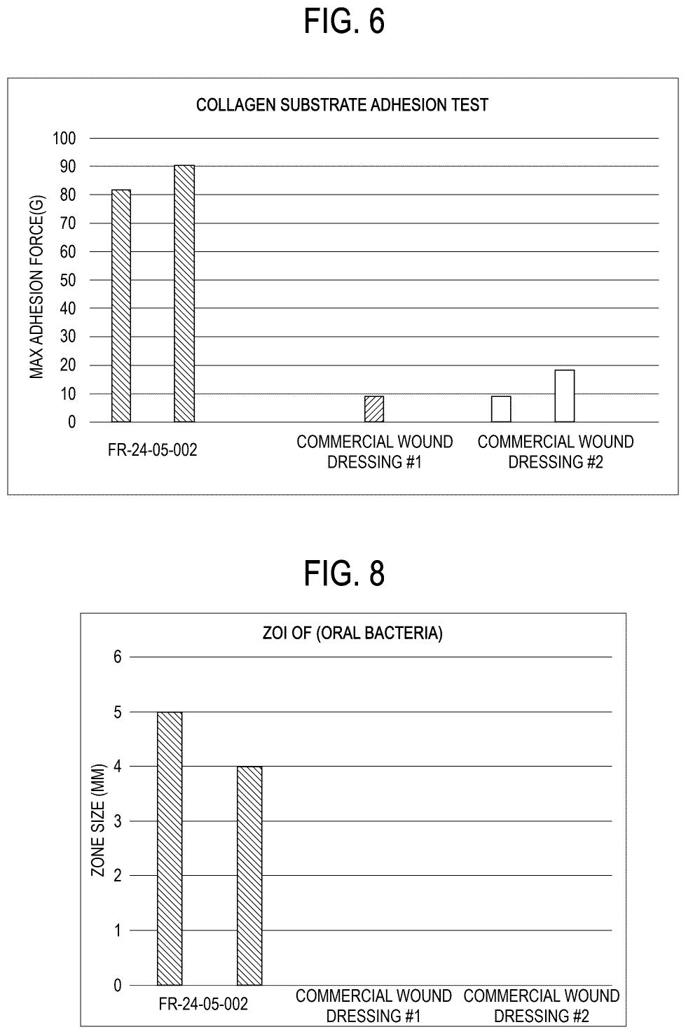

A second adhesion test was conducted using different collagen strip samples (20 mm wide collagen strip vs. 40 mm wide strip in the tests above) and a heavier weight 1000 g to reduce variably in the results and to remove more air from the test system. The results of the collagen strips with the FR-24-05-002 composition were compared to 2 competitive products (commercially available oral hydrogel wound dressing #1 and commercially available oral hydrogel wound dressing #2, a chitosan HCL powder). The results are provided in Table 4, below and graphically in FIG. 6.

TABLE 4
Adhesion testing of Composition FR-24-05-002 vs. Two
Commercially Available Oral Wound Dressing Products

| | | Max Adhesion | Max Adhesion |
|---|---|---|---|
| Test | Product | Force (g) | Force (lbf) |
| 1 | FR-24-05-002 | 82 | 0.18 |
| 2 | | 91 | 0.2 |
| 1 | Commercially | 0 | 0 |
| 2 | available oral | 9 | 0.02 |
| | hydrogel wound | | |
| | dressing #1 | | |
| 1 | Commercially | 9 | 0.02 |
| 2 | available oral | 18 | 0.04 |
| | hydrogel wound | | |
| | dressing #2 | | |

The results show a very strong adhesion force for the present composition as compared to the two commercially available oral hydrogel wound dressings and that the present composition has much higher adhesion to a collagen (tissue substrate) compared to competitive products.

Microbial barrier testing was conducted to determining whether bacteria can penetrate the composition (FR-24-05-002) in place, as a gel and start growing beneath the gel. In this test, hydrated powder was inoculated with bacteria. The hydrated gel was shown to be a “barrier” to microbial penetration.

Polypropylene mesh disks were cut to 8 mm in diameter from a porous mesh (McMaster-Carr 9275T37 polypropylene mesh with a 0.0049 in opening, which allowed for the gel to be contained but provided openings for bacteria to pass through the mesh to an agar plate underneath). The powdered composition coated polypropylene disks were placed onto an agar plate and allowed to hydrate for 30 min at 37° C. The plates were removed from an incubator and 10 μl of bacterial suspension 10⁶ concentration of staphylococcus aureus was applied to the center of the disk. 8 mm disks were used as they provided the surface area necessary to prevent the microbial suspension from rolling off the inoculated surface.

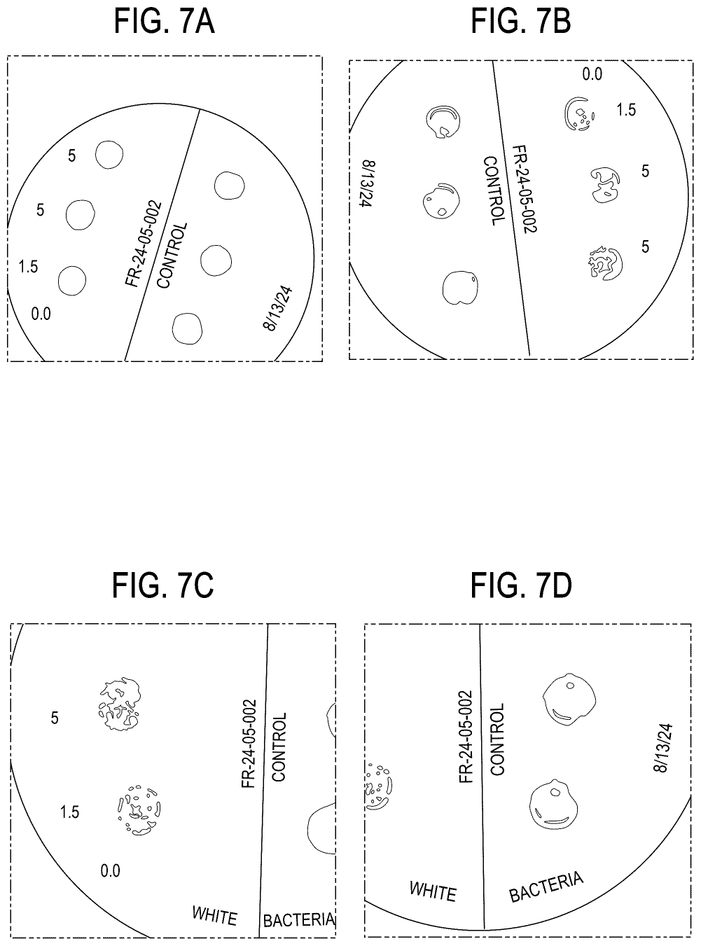

The plates were then incubated at 37° C. for 24 hours. After 24 hours the plates were removed from the incubator and the individual disks were carefully removed from the plate using tweezers (sterilized between each disk, to prevent contamination). The plates were then re-incubated for another 24 hours to observe any bacterial growth under the disks. The results are shown in FIGS. 7A-7D. show that no bacterial growth was observed under the coated disks whereas the control disks (polypropylene mesh disks without powder/gel coating) showed significant growth.

FIG. 7A shows composition (FR-24-05-002) coated disks on the left and control (non-coated) disks on the right on an agar substrate after inoculation. In FIG. 7B, the growth of bacteria (the white) under the disks after removal after 24 hours in the incubator is shown.

The control disks (on the left) show that the un-coated control samples have almost complete coverage of white bacteria under each disk, which shows that bacteria can penetrate the polypropylene mesh. The composition coated disks on the right side show no bacteria growth indicating that the gel was a barrier to microbial penetration (although there is some residual oral powder gel that was transferred to the agar after removal of the disks, under microscopic evaluation it was determined to not be bacteria). FIGS. 7C and 7D are enlarged views showing the control side (FIG. 7C) and the composition coated side (FIG. 7D) after incubation.

Antimicrobial zone of inhibition (ZOI) testing was conducted to determine the extent (e.g., the zone) of antimicrobial effect of the composition. An agar plate was first inoculated with s. aureus bacteria and drops of about 50-100 μl (or for powder applications, disks coated with powder) were placed onto the surface of the plate. The plate was then incubated for 18-24 hours at 37 C.°. After incubation, the plates were removed and observed for areas of non-growth around the original application of the product. This was defined as the ZOI. The larger the zone the greater the antimicrobial effect. Table 5, below, and FIG. 8 show the ZOI for the present composition (FR-24-05-22) and the commercially available oral hydrogel wound dressing #1 and commercially available oral hydrogel wound dressing #2, a chitosan HCL powder. The present composition shows a ZOI of 5 and 4 mm, while the two commercial products show no inhibition to microbial growth.

TABLE 5
Zone of Inhibition (ZOI) Testing of Composition FR-24-05-002
vs. Two Commercially Available Oral Wound Dressing Products

| 3 Experiments | Max ZOI (mm) |
|---|---|
| FR-24-05-002 | 5 |
| | 4 |
| Commercially available oral hydrogel | 0 |
| wound dressing #2 | 0 |
| Commercially available oral hydrogel | 0 |
| wound dressing #1 | |

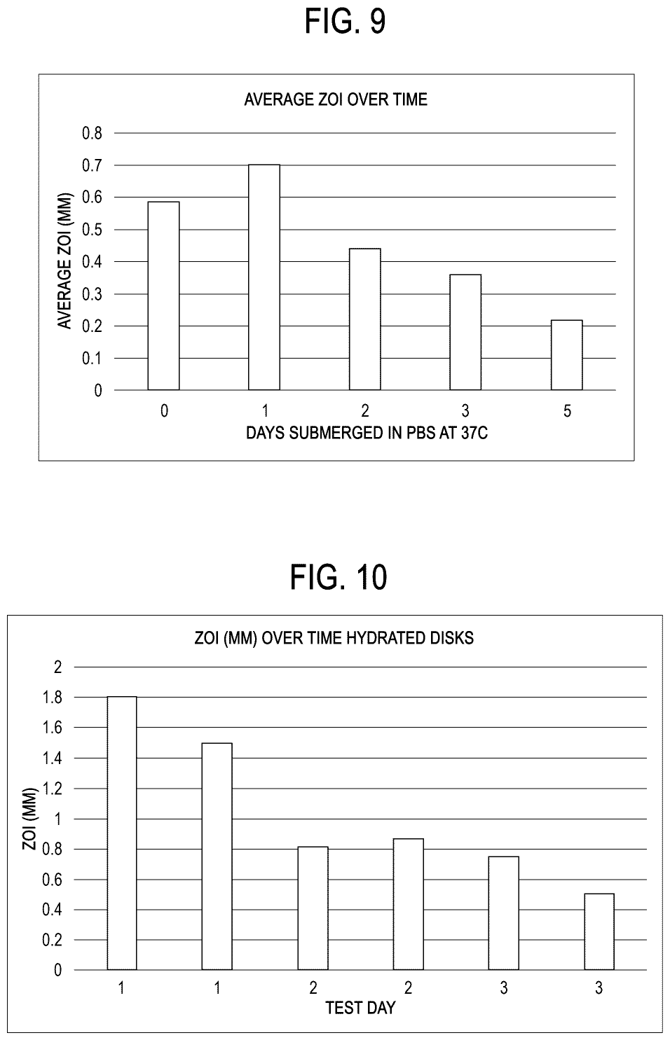

After initial ZOI testing, additional antimicrobial efficacy testing was performed that represented more physiological conditions. The testing was conducted using the present composition (FR-24-05-002) under more realistic in-vivo conditions. Disks were coated with the composition powder and were submerged in 1× phosphate-buffered saline (PBS) solution (10 ml) and incubated for several days at 37° C. The disks were removed every 24 hours for 5 days and plated on bacterial coated plates for ZOI evaluation. The 10 ml dissolution solution (PBS solution) was chosen as a representative or typical volume of saliva that may be present in the oral cavity per day. 10 ml was selected as a worst case scenario and was calculated as an assumption that only 2% of saliva will be exposed to powder per day (based on gum tooth surface area (RSA—root surface area) of 153 mm² (1.53 cm²) compared to oral cavity volume of 111 cm² (about ˜1.5% of the oral cavity volume, rounded up to 2%) is about 8.64 ml of the daily 432 ml of saliva produced per day, and as noted above, which was increased to 10 ml to simulate a worst case scenario. Table 7, below and graphically in FIG. 9, show the result of the simulated physiological conditions ZOI testing.

TABLE 6
Zone of Inhibition (ZOI) Testing of Composition
FR-24-05-002 in Simulated In-Vivo Conditions

| | ZOI (mm) (Hydrated | Average |
|---|---|---|
| Test Day | Disk Samples) | Per Day |
| 0 | 0.70925 | 0.586625 |
| 0 | 0.464 | |
| 1 | 0.926 | 0.7005 |
| 1 | 0.475 | |
| 2 | 0.461 | 0.443 |
| 2 | 0.425 | |
| 3 | 0.2885 | 0.3605 |
| 3 | 0.4325 | |
| 5 | 0.09375 | 0.21875 |
| 5 | 0.34375 | |

Additional test data over 3 days shown in Table 7, below and graphically in FIG. 10, illustrate the antimicrobial activity of the gel over time.

TABLE 7
Zone of Inhibition (ZOI) Testing of Composition
FR-24-05-002 Over 3 Days' Time - Hydrated Disks

| | ZOI (mm) (Hydrated | Average |
|---|---|---|
| Test Day | Disk Samples) | Per Day |
| 1 | 1.8125 | 1.65625 |
| 1 | 1.5 | |
| 2 | 0.8125 | 0.84375 |
| 2 | 0.875 | |
| 3 | 0.75 | 0.625 |
| 3 | 0.5 | |

The limits of the antimicrobial effects were then explored to determine where the ZOI antimicrobial effectiveness drops off. Testing was conducted as the above, but added an additional day (day 4) to the dissolution tests. The results show that the antimicrobial efficacy drops off after 4 days. In certain scenarios, for example, wound healing, this “drop off” may be beneficial as the critical timeframe for cellular remodeling and healing is between 3-4 days.

TABLE 8
Zone of Inhibition (ZOI) Limits Testing of Composition
FR-24-05-002 Over Time - Hydrated Disks

| | ZOI (mm) (Hydrated | Average |
|---|---|---|
| Test Day | Disk Samples) | Per Day |
| 1 | 0.7465 | 0.86 |
| 1 | 0.83 | |
| 1 | 0.9975 | |
| 2 | 1.08 | 0.91 |
| 2 | 0.6625 | |
| 2 | 0.9975 | |
| 3 | 0.6875 | 0.58 |
| 3 | 0.34375 | |
| 3 | 0.7 | |
| 4 | 0 | 0.00 |
| 4 | 0 | |
| 4 | 0 | |

The pH of composition FR-24-05-002 was evaluated and compared to two competitive products (commercially available oral hydrogel wound dressing #2, and commercially available oral hydrogel wound dressing #3—also a chitosan-based dressing), which showed pH values of 3.7 and 5.9, respectively. The results of the pH testing shown below in Table 8 are the average and standard deviation of pH values measured from 15 samples.

TABLE 9
Average pH of Composition FR-24-05-002

| Average pH | STDEV pH | Low pH | High pH |
|---|---|---|---|
| 7.52 | 0.15 | 7.3 | 7.78 |

The chemical composition of the present disclosure can be manufactured in a variety of ways. For example, in one embodiment the chemical composition can be prepared via spray drying the raw materials, such as the polymer base and at least one therapeutic agent, to reduce the particle size and promote rapid hydration. In embodiments, the polymer base and the at least one therapeutic agent can be combined into a homogeneous solution and spray dried together, resulting in the production of a homogeneous powder. In embodiments, separate spray drying of the crosslinker may be necessary to prevent pre-mature crosslinking of the polymer base.

In another embodiment, a retarder such as sodium phosphate, Na3PO4, can be added to the at least one crosslinker to bind ions, for example, calcium ions (Ca2+) that may be released from the at least one crosslinker, such as CaSO4 or CaCl, precipitating as low-soluble calcium phosphate, which can reduce the rate of the reaction and prolong the crosslinking process.

The dry polymer powder and the dry crosslinker powder may be combined to form a homogeneous powder mixture by any suitable means known in the art, such as shaking, mixing, and/or tumbling.

The powder mixture may be placed in a capped applicator contained in a foil/polymer pouch, with or without desiccant, to minimize moisture adsorption and extend shelf life. The application may be made, for example, of a polymer or metal such as stainless steel. The applicator may comprise a plunger operative to push powder out of the applicator when it is depressed. The applicator may be configured for a single, continuous application of the composition.

Referring now to FIG. 1, a process for manufacture and delivery of a chemical composition for oral applications is illustrated. The process begins with parallel dry powder processes. In a first parallel process, the polymer base and the therapeutic agent are hydrated in a polar liquid, such as water, to form a first hydrated solution. Furthermore, in the second parallel process at least one crosslinker is hydrated in a polar liquid, such as water, to form a second hydrated solution. Once hydrated, both the first and second hydrated solutions are independently spray dried, or otherwise dehydrated, to form a first and second dry powder, respectively.

Once formed, the first and second dry powders are combined and placed into a delivery device, such as a syringe, tube, dispenser, etc., and sealed for delivery to a user. Delivery can be performed utilizing known processes, such as spoons, scoops, syringes, funnels, or manually using a gloved finger to the intended area.

Once packaged, a user can retrieve the delivery device from the packaging, utilizing known methods, for application. To apply the formulation, the user can remove a tip, cap, or lid, of the delivery device, and place an applicator tip of the delivery device between the gums and teeth, applying the chemical formulation. The formulation can be spread utilizing the application portion of the delivery device to provide an even coating over a mucosal surface.

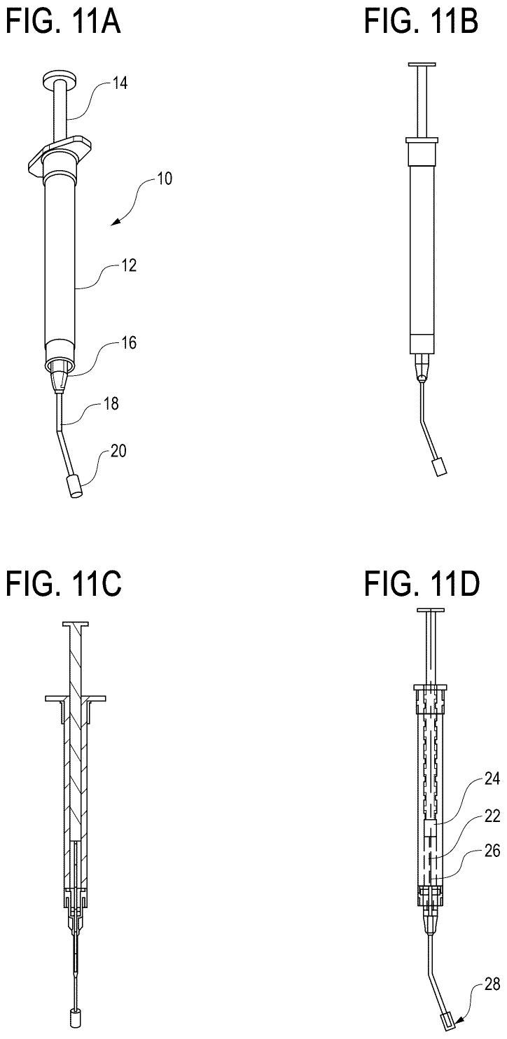

One embodiment of a delivery device 10 is illustrated in FIGS. 11A-11D. Referring to FIGS. 11A and 11B, the device 10, generally, a syringe, includes a body 12, a syringe plunger rod 14, a dispenser hub 16, such as a Luer dispenser hub, a dispenser tip 18, such as a Luer dispenser tip that fastens to the dispenser hub 16, and a cap 20, such as a silicone cap.

The internal portions of the syringe 10 are shown in FIGS. 11C and 11D. The syringe includes a powder (powdered composition) plunger rod 22 and syringe plunger tip 24. Powder (powdered composition 26) may be stored in the dispenser tip 28. It is anticipated that such a delivery device 10 can be a one-time use (disposable) device or can be configured as a reusable (sterilizable/autoclavable) device.

The words “a” or “an” are to be taken to include both the singular and the plural. Conversely, any reference to plural items shall, where appropriate, include the singular. The words “first,” “second,” “third,” and the like may be used in the present disclosure to describe various information, such information should not be limited to these words. These words are only used to distinguish one category of information from another. The directional words “top,” “bottom,” “up,” “down,” “front,” “back,” and the like are used for purposes of illustration and as such, are not limiting. Depending on the context, the word “if” as used herein may be interpreted as “when” or “upon” or “in response to determining.”

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. A powdered composition for oral application of therapeutic agents, comprising:
a base material, at least one therapeutic agent, and a crosslinker,
wherein the composition is formed as a homogenous powder.

2. The powdered composition of claim 1, wherein the base material is one or a combination of a polysaccharide, collagen, chitosan, agar, a thermopolymer, poloxamer, xanthium gum, alginic acid, cellulose, and sodium alginate.

3. The powdered composition of claim 1, wherein the base material is present in a concentration of about 75 percent to about 99 percent by weight of the composition.

4. The powdered composition of claim 3, wherein the base material is present in a concentration of about 90 percent to about 95 percent by weight of the composition.

5. The powdered composition of claim 2, wherein the base material is a polymer.

6. The powdered composition of claim 5, wherein the polymer is one or a combination of polysaccharide, alginic acid, collagen, and chitosan.

7. The powdered composition of claim 1, wherein the therapeutic agent is one or a combination of antimicrobial surfactants, hemostats, antibiotics, and anti-inflammatory agents.

8. The powdered composition of claim 7, wherein the antimicrobial surfactant is cetylpyridinium chloride.

9. The powdered composition of claim 8, wherein the cetylpyridinium chloride is present in a concentration of about 0.010 percent to about 0.10 percent by weight of the composition.

10. The powdered composition of claim 1, wherein the crosslinker is one or a combination of a homo-bifunctional, a hetero-bifunctional, and photoreactive crosslinking agents.

11. The powdered composition of claim 10, wherein the crosslinker is a calcium crosslinker,

12. The powdered composition of claim 11, wherein the calcium crosslinker is one or a combination of calcium sulfate and calcium chloride.

13. The powdered composition of claim 12, wherein the calcium crosslinker is present in a concentration about 1.0 percent to about 10.0 percent by weight of the composition.

14. The powdered composition of claim 13, wherein the calcium crosslinker is present in a concentration of about 5.21 percent by weight of the composition.

15. The powdered composition of claim 1, wherein the crosslinker is one or a combination of a homo-bifunctional, a hetero-bifunctional, and photoreactive crosslinking agents.

16. The powdered composition of claim 15, wherein the homo-bifunctional crosslinker is a divalent metal ion crosslinker.

17. The powdered composition of claim 16, wherein the divalent metal ion crosslinker is one or a combination of calcium sulfate and calcium chloride.

18. The powdered composition of claim 1, wherein the base material is a polymer present in concentration of about 90 percent to about 95 percent by weight of the composition, a crosslinker present in a concentration of about 5 percent to about 10 percent by weight of the composition, and an antimicrobial therapeutic agent present in a concentration of about 0.01 percent to about 0.10 percent by weight of the composition.

19. A powdered composition for oral application of therapeutic agents, comprising:
a base material and a crosslinker,
wherein the composition is formed as a homogenous powder.

20. The powdered composition of claim 19, wherein when subject to a moist anatomical location hydrates into a mucoadhesive or bioadhesive hydrogel.

21. The powdered composition of claim 20, wherein the mucoadhesive or bioadhesive gel crosslinks in-situ into a solid hydrogel.

22. The powdered composition of claim 21, wherein the solid hydrogel forms a barrier to microbial contamination.

23. A delivery device for a powdered composition for oral application of a therapeutic agent, comprises:
a syringe including a body, a syringe plunger rod, a dispenser hub, a dispenser tip, a cap, a composition plunger rod and syringe plunger tip,
wherein the powdered composition is stored in the dispenser tip, and depressing the syringe plunger rod dispenses the powdered composition from the delivery device.

24. The delivery device of claim 23, wherein the delivery device is disposable in its entirety.

25. The delivery device of claim 23, wherein the dispenser hub is a Luer dispenser hub.

26. The delivery device of claim 25, wherein the a dispenser tip is a Luer dispenser tip and fastens to the Luer dispenser hub.
